(19) United States
(12) Patent Application Publication
(10) Pub. No.: US 2021/0321937 A1
(43) Pub. Date: Oct. 21, 2021

(54) BIRTH DELIVERY MAGNETIC TRACKING SYSTEM

(71) Applicant: TRIG MEDICAL LTD., Nesher (IL)

(72) Inventor: Dan AMIT, Kiryat Motzkin (IL)

(21) Appl. No.: 17/271,810

(22) PCT Filed: Aug. 23, 2019
(86) PCT No.: PCT/IB2019/057102

§ 371 Date: Feb. 26, 2021

Related U.S. Application Data
(60) Provisional application No. 62/724,726, filed on Aug. 30, 2018.

(51) Int. Cl. A61B 5/00

(52) U.S. Cl. CPC A61B 5/4362; A61B 5/6892; A61B 2562/046; A61B 2562/0223; A61B 5/6823

(57) ABSTRACT

A method of tracking progress of labor includes placing a magnetic field sensor array (14), which includes an array of magnetic sensors (16), close to a woman who is carrying a fetus, placing tracking devices (20, 22, 23, 24) at positions relative to the fetus, generating a magnetic field from each of the tracking devices (20, 22, 23, 24), each of the magnetic fields being unique to a particular one of the tracking devices (20, 22, 23, 24), sensing the magnetic fields of the tracking devices (20, 22, 23, 24) with the magnetic field sensor array (14), analyzing sensed magnetic fields of the tracking devices (20, 22, 23, 24) to identify positions of the tracking devices (20, 22, 23, 24) with respect to the fetus, and using the positions of the tracking devices (20, 22, 23, 24) with respect to the fetus to determine progress of labor of the woman.

### FIELD OF THE INVENTION

The present invention relates to birth delivery devices, and particularly to a magnetic tracking system for tracking progress of labor and birth delivery.

### BACKGROUND OF THE INVENTION

A number of other physiological conditions of the mother and baby during labor can be monitored in order to determine the progress of labor. These conditions include: (1) effacement (the thinning out of the cervix that occurs before and during the first stage of labor); (2) cervical dilatation (the increase in size of the cervical opening); (3) position of the cervix (the relation of the cervix to the vaginal axis, normally the fetal head); (4) station (the level of a predetermined point of the fetal presenting part with reference to the mother's pelvis), (5) position of the head which describes the relationship of the head to the pelvis and (6) and presentation which describes the part of the fetus (such as brow, face or breech) at the cervical opening.

Systems exist for monitoring the progress of labor. For example, U.S. Pat. Nos. 6,200,279 and 6,669,653 to Paltieli, incorporated herein by reference in their entirety, describe methods and apparatus for monitoring the progress of labor. Based on these and other patents, Trig Medical Ltd. has developed the LABORPRO (LP) tracker.

The LP tracker includes a main electronic module, a magnetic field transmitter and position sensors, from Ascension Technologies (ATC).

The magnetic field transmitter includes two types of transmitters: a flat transmitter positioned under the patient bed's mattress and a cubical transmitter mounted on a mechanical arm.

The position sensors include passive coils which sense the magnetic field that is generated by the magnetic field transmitter. A 3 degree-of-freedom (DOF) disposable sensor has one coil while a 6 DOF sensor has 3 coils. These coils are incorporated in passive sensor tips. The sensed signals are amplified by an electronic preamplifier and connected to the main electronic module. Based on these signals, the main electronic module identifies each sensor's spatial location and orientation.

There are Three Position Sensors:

Disposable back sensor (“back sensor”): This sensor (1.8-mm in diameter) is attached to the patient's back by a sticker overlying the L5 spinous process, and remains in place during the entire monitoring period. This sensor is used as a position reference sensor during labor. This sensor provides 3 DOF positional data.

Ultrasound sensor: This sensor (8-mm in diameter) is attached to the abdominal probe of an off-the-shelf ultrasound system. This probe is pre-calibrated allowing precise mapping of each pixel in the image created by the ultrasound probe to the transmitter's 3D operating volume. The data provided by the attached positional sensor provides the spatial position and orientation (6 DOF) of the probe, facilitating the relative 3D positioning of all pixels in the ultrasound image.

Finger sensor: This sensor (1.3-mm in diameter), is used for ruler-like measurement of the distance between different points of interest. This sensor provides 3 DOF positional data.

### SUMMARY OF THE INVENTION

The present invention seeks to provide a novel magnetic tracking system for tracking (monitoring) the progress of labor (birth delivery), as is described more in detail hereinbelow. In addition, the magnetic tracking system may be used to guide needles in surgical procedures.

There is provided in accordance with an embodiment of the present invention a method of tracking progress of labor including placing a magnetic field sensor array, which includes an array of magnetic sensors, close to a woman who is carrying a fetus, placing tracking devices at positions relative to the fetus, generating a magnetic field from each of the tracking devices, each of the magnetic fields being unique to a particular one of the tracking devices, sensing the magnetic fields of the tracking devices with the magnetic field sensor array, analyzing sensed magnetic fields of the tracking devices to identify positions and orientations of the tracking devices with respect to the fetus, and using the positions and orientations of the tracking devices with respect to the fetus to determine progress of labor of the woman.

In accordance with an embodiment of the invention the magnetic field sensor array includes a plate placed under or above a mattress of a delivery bed on which the woman is lying.

In accordance with an embodiment of the invention the magnetic field sensor array includes a plate placed near, but not on, a delivery bed on which the woman is lying.

In accordance with an embodiment of the invention at least one of the tracking devices provides spatial information in three degrees of freedom

In accordance with an embodiment of the invention at least one of the tracking devices provides spatial information in six degrees of freedom.

In accordance with an embodiment of the invention at least one of the tracking devices includes a lower back or pelvic sensor attached to or near a lower back, spinous process or pelvis of the woman.

In accordance with an embodiment of the invention at least one of the tracking devices includes an ultrasonic sensor.

In accordance with an embodiment of the invention at least one of the tracking devices includes an inclinometer.

In accordance with an embodiment of the invention at least one of the tracking devices includes finger sensor.

In accordance with an embodiment of the invention using the positions of the tracking devices with respect to the fetus to determine progress of labor of the woman includes determining a station of a head of the fetus and/or determining a position and angular orientation of a head of the fetus.

### BRIEF DESCRIPTION OF THE DRAWINGS

The invention is herein described, by way of example only, with reference to the accompanying drawings, wherein:

FIG. 1 is a schematic illustration of a magnetic tracking system for monitoring labor progress, constructed and operative in accordance with an embodiment of the present invention;

FIG. 2 is a simplified block diagram of the magnetic tracking system; and

FIG. 3 is a simplified flow chart of a method of using the magnetic tracking system, in accordance with an embodiment of the present invention.

### DETAILED DESCRIPTION OF THE INVENTION

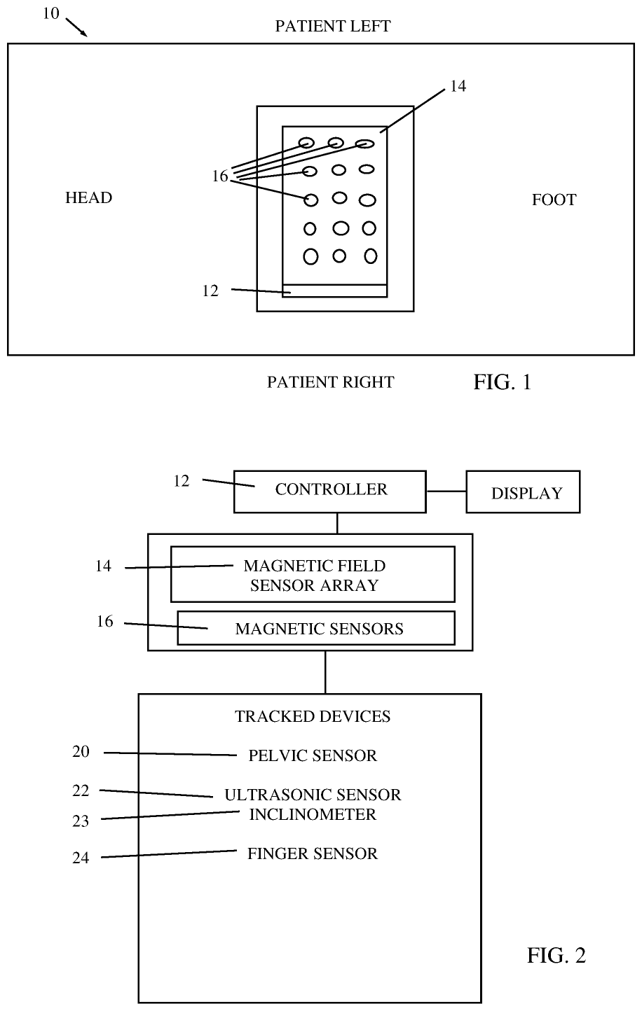

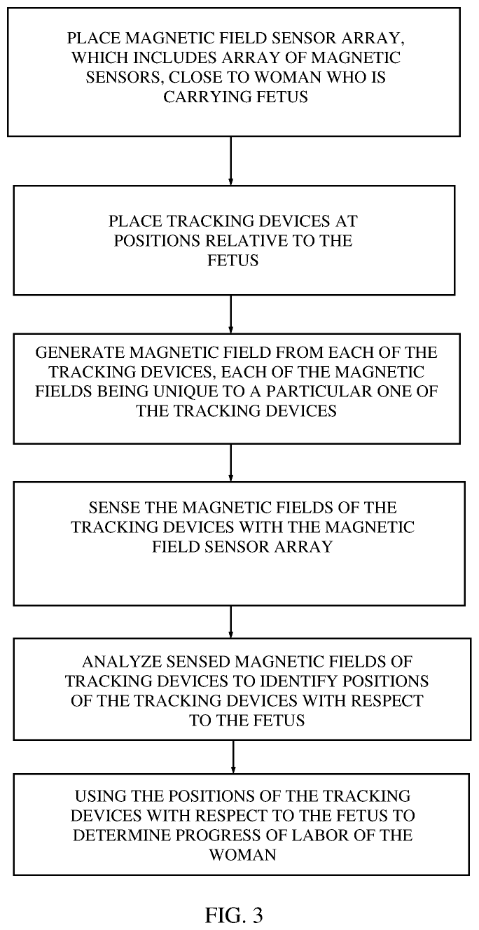

Reference is now made to FIGS. 1 and 2, which illustrate a magnetic tracking system 10, constructed and operative in accordance with an embodiment of the present invention.

Magnetic tracking system 10 includes a main electronics module (also called controller or processor) 12 and a magnetic field sensor array 14, which may be in the form of a plate. Magnetic field sensor array 14 replaces the flat transmitter of the prior art tracker and provides significantly different and improved functionality.

Magnetic tracking system 10 also includes tracked devices, which replace the position sensors of the prior art tracker. In the prior art, the sensors are passive. In contrast, in the present invention, the tracked devices generate a magnetic field which is sensed by magnetic field sensor array 14. Passive devices are detrimentally affected by any noise in the signals; this problem is greatly diminished and may be insignificant with the active devices of the invention. The processing needed to process information from the passive devices is also much more cumbersome and takes more time than the processing with active devices.

The tracked device may be a magnet or a coil or a set of coils generating a magnetic field.

The magnetic field sensor array 14 may be, without limitation, a flat rectangular plate (or other shapes) placed under or above the mattress of the delivery bed, or positioned on the bedside. The sensor array 14 may be constructed, without limitation, as an electronic board with a built-in array of magnetic sensors 16.

Sensors 16 sense the magnetic field generated by the tracked devices. Based on the sensed magnetic field, magnetic field sensor array 14 identifies the position of the tracked devices. The magnetic sensors 16 are in communication with controller 12, which processes the sensed information and provides displays of the labor progress as sensed by the tracked devices, such as but not limited to, the location of the fetal presenting part with respect to a predetermined point on the mother's pelvic bones, effacement, cervical dilatation, cervical position and many more.

One of the tracked devices may be a lower back or pelvic sensor 20. Table 2 gives non-limiting parameters of sensor 20.

Lower back sensor 20 may be a disc magnet attached to the patient's back by a sticker overlying the L5 spinous process, which remains in place during the entire monitoring period. Lower back sensor 20 is used as a position reference sensor during labor and provides 3 DOF positional data. Lower back sensor 20 generates a magnetic field that is sensed by magnetic field sensor array 14.

One of the tracked devices may be an ultrasonic sensor 22. Table 3 gives non-limiting parameters of sensor 22.

Ultrasonic sensor 22 may be attached to the abdominal probe of a standard ultrasound system on a known location allowing precise mapping of each pixel in the image created by the ultrasound probe to the plate's 3D operating volume. Sensor 22 provides the spatial position and orientation (6 DOF) of the probe, facilitating the relative 3D positioning of all pixels in the ultrasound image. Ultrasonic sensor 22 generates a magnetic field that is sensed by magnetic field sensor array 14. Based on this sensed magnetic field, magnetic field sensor array 14 identifies the position of ultrasonic sensor 22 (3 DOF). In order to provide the orientation data, an inclinometer 23 (e.g., IMU—inertial measurement unit) may be attached to or may be part of the ultrasound sensor 22.

The data provided by the inclinometer may be used to control the current of each coil, which enables controlling the direction of the magnetic field.

The ultrasound sensor 22 may provide the station of the fetus (how far the fetal head has descended relative to the mother's pelvis; if the fetal head is level with the ischial spines, the fetal station is zero). The inclinometer 23 provides information on the spatial position of the ultrasound probe allowing determination of position and angular orientation of the fetal head (e.g., angle of the fetal head relative to the pelvis) and calculation of needle trajectory before and during insertions.

One of the tracked devices may be a magnetic finger sensor 24. Table 4 gives non-limiting parameters of finger sensor 24.

Sensor 24 is a magnet which is attached to the finger tip of the user and may be placed under a glove. Sensor 24 provides ruler-like measurements of the distance between two points of interest by touching these points, and determines their spatial locations. Sensor 24 provides 3 DOF positional data.

All positional data may be transferred to the main unit controller 12 via USB connection or wirelessly.

Any errors originated from magnet tilt angle may be corrected with appropriate error correction methods.

The magnets or magnetic sensors may be placed on any of the tracking devices (for example, any ultrasonic probe or any other device) in a known, predetermined spatial position and orientation. The known spatial position and orientation may be used for calibration of the system so that the spatial position and orientation of the tracked device may be monitored by the system. Any interface may be used for data transfer, such as USB, HDMI and many others.

TABLE 1
The Plate Electronic Board General Requirements

| Parameter | Value | Features |
|---|---|---|
| Plate Board Height | <10 mm | |
| Operating Voltage | 5VDC | By external medical |
| | | power supply. |
| | | In each |
| | | case <=2VDC |
| Interface #1 | USB | |
| Interface #2 | Wireless | May be connectable |
| | | via Wi-Fi |
| Sensing Area | 300 × 400 mm | 300 mm along head |
| | | to foot direction. |
| | | 400 mm along left |
| | | to right direction. |
| Placement | All additional components | |
| of additional | should be places on one | |
| electronic | side of the sensing area | |
| components | which is along the width of | |
| | the bed | |

TABLE 2
Maternal Back Magnet Tracking Requirements

| Parameter | Value | | Features |
|---|---|---|---|
| Device Type | Magnetic Disc | | Neodymium N52 magnets |
| Device Diameter | 15 mm-26 mm | | |
| Device Thickness | <1.6 | mm | |
| Height above plate (op. 1) | <50 | mm | When the plate is placed |
| | | | above the mattress |
| Height above plate (op. 2) | <120 | mm | When the plate is placed |
| | | | under the mattress |
| X Axis Accuracy | <2 | mm | X Axis is along the bed |
| Y Axis Accuracy | <5 | mm | |
| Height Accuracy | <6 | mm | |
| Measurement Response | <50 | ms | |
| Time | | | |

| | |
|---|---|
| Measurement Type | 3 DOF |

TABLE 3
Ultrasound Probe Tracking Requirements

| Parameter | Value | | Features |
|---|---|---|---|
| Device Type | Magnet or 3 Orthogonal | | |
| | Coils + IMU or 3 axis | | |
| | Inclination device | | |
| Device Size | <30 × 30 × 20 mm | | WxDxH |
| Height above plate | <400 | mm | |
| X Axis Accuracy | <2 | mm | |
| Y Axis Accuracy | <5 | mm | |
| Height Accuracy | <6 | mm | X Axis is along the bed |
| Angle Accuracy | <0.5° | | |
| Measurement | <50 | ms | |
| Response Time | | | |
| Measurement | 6 DOF | | Position 3DOF-Using |
| Type | | | magnet or coils |
| | | | Orientation 3DOF-Using |
| | | | IMU or inclinometers |

TABLE 4
Finger Magnet Tracking Requirements

| Parameter | Value | | Features |
|---|---|---|---|
| Device Type | Magnet/Coils | | |
| Device Dimensions | <4 × 4 × 10 mm | | |
| Height above plate | 30-200 mm | | |
| X Axis Accuracy | <2 | mm | X Axis is along the bed |
| Y Axis Accuracy | <2 | mm | |
| Height Accuracy | <5 | mm | |
| Measurement | 50 | ms | |
| Response Time | | | |

| | |
|---|---|
| Measurement Type | 3 DOF |

1. A method of tracking progress of labor comprising:
placing a magnetic field sensor array, which comprises an array of magnetic sensors, adjacent to a woman who is carrying a fetus;
placing tracking devices at different respective positions relative to said fetus;
generating magnetic fields from said tracking devices, respectively, each of said magnetic fields being unique to one of said tracking devices;
sensing said magnetic fields of said tracking devices with said magnetic field sensor array;
analyzing sensed magnetic fields of said tracking devices to identify respective positions and orientations of said tracking devices with respect to said fetus; and
using said respective positions and orientations of said tracking devices with respect to said fetus to determine progress of labor of the woman.

2. The method according to claim 1, wherein said magnetic field sensor array comprises a plate placed under the woman.

3. The method according to claim 1, wherein said magnetic field sensor array comprises a plate that is not on a surface on which the woman is lying or sitting.

4. The method according to claim 1, wherein at least one of said tracking devices provides spatial information in three degrees of freedom.

5. The method according to claim 1, wherein at least one of said tracking devices provides spatial information in six degrees of freedom.

6. The method according to claim 1, wherein at least one of said tracking devices comprises a lower back or pelvic sensor attached to or near a lower back, spinous process or pelvis of the woman.

7. The method according to claim 1, wherein at least one of said tracking devices comprises an ultrasonic sensor.

8. The method according to claim 1, wherein at least one of said tracking devices comprises an inclinometer.

9. The method according to claim 1, wherein at least one of said tracking devices comprises finger sensor.

10. The method according to claim 1, wherein using said respective positions of said tracking devices with respect to said fetus to determine progress of labor of the woman comprises determining a station of a head of said fetus.

11. The method according to claim 1, wherein using said positions of said tracking devices with respect to said fetus to determine progress of labor of the woman comprises determining a position and angular orientation of a head of said fetus.

12. A tracking system, comprising:
a plurality of magnetic field generators, wherein each magnetic field generator of the plurality of magnetic field generators is configured to generate a respective magnetic field which is unique to the magnetic field generator;
a plate having a plurality of built-in magnetic sensors, wherein the plurality of magnetic sensors are configured to sense magnetic fields generated, respectively, by the plurality of magnetic field generators; and
a processor configured to:
communicate with the plurality of magnetic sensors to receive sensed information describing the magnetic fields sensed by the plurality of magnetic sensors;
process the sensed information to determine respective positions and orientations of the plurality of magnetic field generators;
determine, based on the respective positions and orientations of the plurality of magnetic field generators, information which describes progress of labor associated a woman's delivery of a fetus.

13. The tracking system of claim 12, wherein the plurality of magnetic field generators include a plurality of magnets.

14. The tracking system of claim 12, wherein the plurality of magnetic field generators include a plurality of conductive coils that, when activated, are configured to generate the magnetic fields.

15. The tracking system of claim 12, wherein the plurality of magnetic sensors are arranged as an array.

16. The tracking system of claim 12, wherein the processor is configured, when at least one magnetic field generator of the plurality of magnetic field generators is part of or placed on a pelvic sensor, to determine a location of a part of the fetus with respect to the woman's pelvic bones.

17. The tracking system of claim 16, wherein the processor is configured to determine, based on the magnetic fields sensed by the plurality of magnetic sensors, at least one of: a location of a fetal presenting part of the fetus with respect to the woman's pelvic bones, or an angle of a head of the fetus relative to the woman's pelvis.

18. The tracking system of claim 12, wherein the processor is configured to determine, based on the magnetic fields sensed by the plurality of magnetic sensors, cervical dilation associated with the labor.

19. The tracking system of claim 12, wherein the processor is configured, when at least one magnetic field generator of the plurality of magnetic field generators is part of or placed on an ultrasound probe, to map pixels in an image created by the ultrasound probe to a three-dimensional volume of the plate.

20. A tracking system, comprising:
a plate having a plurality of built-in magnetic sensors and having a processor,
wherein the plurality of magnetic sensors are configured to sense magnetic fields generated, respectively, by a plurality of magnetic field generators, and
wherein the processor is configured to:
communicate with the plurality of magnetic sensors to receive sensed information describing the magnetic fields sensed by the plurality of magnetic sensors;
process the sensed information to determine respective positions and orientations of the plurality of magnetic field generators; and
determine, based on the respective positions and orientations of the plurality of magnetic field generators, information which describes progress of labor associated with a fetus.
